(19) United States
(12) Patent Application Publication
(10) Pub. No.: US 2002/0055111 A1
(43) Pub. Date: May 9, 2002

(54) Three-dimensional probe carriers

(76) Inventors: Shiping Chen, Rockville, MD (US); Anthony Chen, Sunnyvale, CA (US); Yuling Luo, Castro Valley, CA (US)

Correspondence Address: Charles D. Holland, Morrison & Foerster LLP, 755 Page Mill Road, Palo Alto, CA, 94304, US

(21) Appl. No.: 09/938,798

(22) Filed: Aug. 24, 2001

Related U.S. Application Data
(60) Provisional application No. 60/227,896, filed on Aug. 25, 2000.
(60) Provisional application No. 60/292,069, filed on May 17, 2001.

(51) Int. Cl. C12Q 1/68; C12M 1/34

(52) U.S. Cl. 435/6; 435/287.2

(57) ABSTRACT

The inventions disclosed herein provide a novel 3-dimensional arrangement of probes on a probe carrier with concomitant increases in the economy of reagents, compactness and readability of the probe carriers. Novel methods of fabricating probe carriers are also disclosed. Techniques for hybridization assays and reading results of such assays are also presented.

### CROSS-REFERENCE TO RELATED APPLICATIONS

This application claims the benefit of priority to U.S. Ser. No. 60/227,896, entitled “GeneHive,” filed Aug. 25, 2000, and U.S. Ser. No. 60/292,069, entitled “Three-Dimensional Probe Carriers,” filed May 17, 2001, each of which is incorporated by reference in its entirety herein as if fully put forth below. This application is also related to PCT application entitled “Three-Dimensional Probe Carriers,” applicant GenoSpectra, Inc., inventors as listed above, Attorney Docket No. 473532000540, which is filed on even date herewith and which is also incorporated by reference in its entirety as if fully set forth below.

### TECHNICAL FIELD OF THE INVENTION

This application relates to architecture of microarrays. Specifically, this application relates to three-dimensional probe-carrier structures. More specifically, this application relates to porous three-dimensional probe-carrier structures.

### BACKGROUND OF THE INVENTION

A DNA microarray refers to an array of known DNA samples (“probes”) immobilized as microscopic spots at predefined positions on a solid substrate.

A vast number of chemical and biological analyses involve carrying out tests on a very large number of chemical or biological samples. Examples include gene expression analysis, SNP identification, genotyping and drug screening. The microarray is a powerful tool. It is capable of dramatically boosting the efficiency of biochemical investigation by conducting these tests in a massively parallel fashion.

Existing microarrays consist of a large number of unique, known chemical or biological samples (probes) immobilized as microscopic spots on a flat support. Another biochemical sample (target), often with certain unknown characteristics, is brought to interact with all the probes on the array. The results of these interactions are read out by fluorescent, chemiluminescent or other radiation-based indicators.

With the existing microarray format, the probes have to be immobilized in order to prevent cross talk between different biological/chemical reactions carried out on a flat surface. Chemical/biological reactions with immobilized probes, however, are typically slow, inefficient and, in many cases, invalid. There are several application areas where fluid phase reactions are of particular importance.

The first application area is proteomics. It is very difficult to apply the conventional microarray format directly to proteins because most proteins, once dried, will denature making it very hard to immobilize protein based probes on to a substrate. As a result, the conventional microarray format cannot be readily applied to proteins for proteomics applications.

The second application area is Polymerase Chain Reaction (PCR). PCR is a powerful DNA amplification technique that mimics nature's way of replicating DNA. First described in 1985, PCR has been adopted as an essential research tool because it can take a minute sample of genetic material and duplicate enough of it for study. PCR has been used to identify the remains of Desert Storm casualties, to analyze prehistoric DNA, to diagnose diseases and to help make identifications in police investigations.

DNA is most often found as a double-stranded molecule, twisted as a helix, in which each strand complements the other. PCR starts with the DNA sample, which is put in a reaction tube along with primers (short, synthetic pieces of single-stranded DNA that exactly match and flank, from each side of the sequence, the stretch of DNA to be amplified), deoxynucleotide triphosphates (dNTPs, the building blocks of DNA), buffers and a heat-resistant enzyme (polymerase). Heating the mixture separates the “template” strands of DNA. Then, at varying temperatures, the rest of the components in the mixture spontaneously organize themselves, building a new complementary strand for each original. At the end of each cycle the DNA count has doubled. If you start with one DNA molecule, at the end of 30 cycles (only a few hours later) there will be about a billion copies.

Because of the sample amplification power of the PCR, it has been used to detect the existence of certain DNA in minute biological samples. One of such technique involves the use of a fluorogenic probe in the 5′ nuclease assay, which combines PCR amplification and detection into a single step. In the 5′ nuclease assay as first described by Holland et al (1991, 1992), an additional hybridization probe complementary to the target sequence is added to the PCR reaction mixture. The probe consists of an oligonucleotide with a reporter and quencher dye attached. In the intact probe, proximity of the quencher reduces the fluorescence signal observed from the reporter dye, most likely due to Forster resonance energy transfer (FRET) (Förster 1948, Lakowicz 1983). During PCR, if the target of interest is present, the probe anneals specifically between the forward and reverse primer sites. The nucleolytic activity of the polymerase cleaves the probe, which results in an increase in the fluorescence intensity of the reporter dye. This process occurs in every cycle and does not interfere with the accumulation of PCR product. The measurement of this fluorescence increase during the thermal cycling of PCR, provides an extremely sensitive, “real-time|” detection of PCR product accumulation.

In the PCR based biological reactions described above, the primers and fluorogenic probe have to be vastly abundant and highly mobile. At present, all these reactions are carried out in fluid phase. A typical instrument available today is the TaqMan produced by Applied Biosystems Inc. in 96-well microtiter plate format. In comparison with existing microarray technologies, PCR based detection provides greatly improved sensitivity and specificity and is capable of providing accurate quantitative measurements. However, it can only conduct a small number of detections at the same time, which is vastly inadequate in dealing with today's genomic applications such as gene expression, SNP detection/analysis. The existing microarray technology, on the other hand, provides massive parallel detection capability. Its inferior sensitivity and specificity, however, confines it mostly to qualitative tests.

The third application area where fluid phase reactions are crucial is drug screening. In order to identify potentially useful drug components, a specific assay has to be tested against a very large established library of chemical compounds. In each test, a mixture of target assay, enzyme and ligand is labeled with fluorescent material and brought into contact with a specific compound in fluid phase. An effective chemical compound will quench the fluorescence. Thus a reduction in fluorescent emission is served as an indicator to the effectiveness of the compound. Because a typical library of chemical compounds consists of several million compounds, it is most desirable to carry out such a screening process in a massively parallel fashion.

The GeneHive microarray format describe in this document enables fluid phase chemical/biological reactions to be carried out in a massively parallel fashion. It can serve as a basic platform for protein based microarray and will be an enabling tool for proteomics. It facilitates PCR on microarray format. This combines the power of PCR and microarray and greatly simplifies the processing steps in molecular investigations. Finally, GeneHive format can be readily adapted for screening millions of chemical compounds for drug discovery.

In addition to the fluid phase microarrays discussed above, the GeneHive, and in addition, the GenePillar described here can also be used in the same manner as conventional microarrays, where probes are immobilized on either the inner walls of the holes in the GeneHive substrate, or the outer walls of the pillars on GenePillar. The unique three-dimensional structure of these two microarray formats provide much large surface area for probe binding, which helps to enhance signal to noise ratio of the microarray.

### SUMMARY OF THE INVENTION

This invention includes two new three dimensional porous probe-bearing substrate formats as well as methods and apparatus for the low-cost, high throughput fabrication of such probe-bearing substrates. This invention further includes methods and apparatus of using these formats for parallel fluid or solid phase biological/chemical tests.

In one aspect of the invention, a 3-dimensional internal probe-carrier for binding a target molecule to a probe is provided, comprising a solid support member having a first and a second surface; at least one discrete through well on the solid support, the well comprising an elongated bore structure traversing the solid support from the first to the second surface and defined by at least one inner side wall, wherein each well is individually identifiable by its position on the solid support; and at least one specific probe molecule attached to a discrete location on an inner side wall of the well.

In another aspect of the invention, a 3-dimensional internal probe-carrier for binding a target molecule to a probe is provided, comprising a solid support member having a first and a second surface; at least one discrete through well on the solid support, the well comprising an elongated bore structure traversing the solid support from the first to the second surface and defined by at least one inner side wall, wherein each well is individually identifiable by its position on the solid support; a light conducting region surrounding each well; and at least one specific probe molecule contained within the space defined by the through-well.

In another aspect of the invention, a 3-dimensional internal probe-carrier for binding a target molecule to a probe is provided, comprising a solid support member having a first and a second surface; at least one discrete through well on the solid support, the well comprising an elongated bore structure traversing the solid support from the first to the second surface and defined by at least one inner side wall, wherein each well is individually identifiable by its position on the solid support; and at least two different probe molecules immobilized on discrete locations on the inner side wall of each through well.

In another aspect of the invention, a 3-dimensional internal probe-carrier for binding a target molecule to a probe is provided, comprising a solid support member which comprises an optical fiber having a first and a second surface at least one discrete through well on the solid support, the well comprising an elongated bore structure traversing the solid support from the first to the second surface and defined by at least one inner side wall, and wherein each well is individually identifiable by its position on the solid support.

A plurality of different probes may be attached to the inner side walls of the solid support at a density exceeding 100 different probes per square millimeter wherein each discrete region such as a microwell or channel of a capillary individually contains a unique probe.

This invention also relates to a method of hybridizing a target molecule to a 3-dimensional internal probe-carrier by enabling a flow of a hybridization fluid containing the target molecule across and through the elongated bore structure such that the target molecule is able to contact the probe.

A method of reading a hybridization signal on a 3-dimensional internal probe-carrier is provided, comprising providing a target molecule whose interaction with a matching probe on the wall of the through well generates a detectable level of optical signal (e.g., Fluorescence Resonance Energy Transfer); providing an optical waveguide within the through well; allowing the target molecule to interact with the probe on the wall; and reading the optical signal associated with the interaction, wherein the optical signal is transmitted through the optical wave guide to an opening of the well towards an optical reader.

In a preferred embodiment, the method comprises the steps of providing at least one fluorescent tags on the target molecule; providing an optical waveguide within the through well by coating a light reflective layer on the inner side wall of the through well; exciting a fluorescent label on a hybridized target molecule by providing an excitation light coupled into the waveguide such that the light is multiply reflected by the light reflective layer on the inner side wall; reading an emitted fluorescent light emitted by the excited label on the target, wherein the emitted fluorescent light is multiply reflected by the light reflective layer on the inner side wall and guided through an opening of the well towards an optical reader.

A method of fabricating a 3-dimensional internal probe-carrier is provided comprising providing a tube preform; creating an optical waveguide around the through well by providing a light guiding region around the through well bore; extruding one or more preforms such that each individual preform is reduced to a first predetermined diameter; treating the inner surface of the preforms for attachment of probes; cutting the preforms into capillaries of a predetermined length; introducing at least one probe into a capillary; freezing the capillary containing the probe (or attaching the probe to an inner wall of the capillary) and forming a bundle comprising a plurality of the probe-containing capillaries by attaching the capillaries to one another by epoxy or other adhesive; and cutting the preform bundles into chips or pins.

In another aspect of the invention, a 3-dimensional external probe-carrier for binding a target molecule to a probe is provided, comprising a solid support member having a first surface at least one discrete pillar on the first surface of the solid support, the pillar comprising an elongated core and defined by at least one exterior side wall, wherein each pillar is individually identifiable by its position on the solid support and at least one specific probe molecule attached to the exterior side wall of the pillar.

This invention relates to a method of hybridizing a target molecule to a probe comprising providing a solid support member having a first surface comprising at least one discrete pillar on the first surface of the solid support, the pillar comprising an elongated core and defined by at least one exterior side wall, further wherein each pillar is individually identifiable by its position on the solid support; providing at least one specific probe molecule attached to the exterior side wall of the pillar; and contacting the probe with a hybridization fluid containing the target molecule.

This invention relates to a method of reading a hybridization signal on a 3-dimensional external probe-carrier comprising providing a target molecule whose interaction with a matching probe on the pillar generates a detectable level of optical signal (e.g., Fluorescence Resonance Energy Transfer); providing a pillar with an optical waveguide in it; allowing the target molecule to interact with the probe on the pillar; and reading the optical signal associated with the interaction, wherein the optical signal is transmitted by the waveguide within the pillar and guided towards an upper or lower surface in the direction of an optical reader.

In a preferred embodiment, this method comprises providing at least one fluorescent tags on the target molecule; providing a pillar manufactured of a transparent material with an optical refractive index higher than that of the surrounding medium such that an optical waveguide is created within the pillar; exciting a fluorescent label on a hybridized target molecule by providing an excitation light coupled into the waveguide such that the light is multiply reflected by the optical waveguide within the pillar; reading an emitted fluorescent light emitted by the excited label on the target, wherein the emitted fluorescent light is multiply reflected by the optical waveguide within the pillar and guided towards an upper or lower surface in the direction of an optical reader.

This invention relates to a method of fabricating a 3-dimensional external probe-carrier comprising providing an optical fiber comprising a core and an outer layer affixing one or more probes to the outer surface of the fiber such that the location of each probe is determinable; attaching one end of each of one or more fibers to a solid support; coating each probe-attached fiber with a removable, protective layer such that the fiber is held in place; attaching an end of each of a plurality of fibers to one another such that one end of the fibers form a bundle while the other loose end is identifiable by an optical reader, and establishing the identity of each fiber in the bundled end; and cutting the bundle into individual pillars and removing the protective layer, wherein the identity of each fiber in the pillar is established.

This invention relates to a method of fabricating a 3-dimensional external probe-carrier comprising: providing an optical fiber comprising a core and an outer layer; affixing one or more probes to the outer surface of the fiber such that the location of each probe is determinable; holding a plurality of the fibers in an orderly matrix by use of a guide plate; coating with a removable, protective layer such that the matrix of fibers is held in place; cutting the matrix into individual pillars, wherein the identity of each fiber in the pillar is established; attaching an end of each of the pillars to a solid substrate; and removing the protective layer.

### BRIEF DESCRIPTION OF THE DRAWINGS

FIG. 1 illustrates one embodiment of a configuration of the 3-dimensional internal probe-bearing substrate.

FIG. 2 illustrates one embodiment of an use of the 3-dimensional probe carrier as a fluid microarray.

FIG. 3 illustrates the steps in one method of fabrication of the honeycomb embodiment of the 3-dimensional probe carrier.

FIG. 4 illustrates the application of target fluid to the 3-dimensional probe carrier.

FIG. 5 shows a device to promote contact between sample and probes in the 3-dimensional internal probe carrier.

FIG. 6 schematically demonstrates a specific embodiment of a device to promote contact between sample and probes in which sample is forced through the honeycomb by means of deformation of two diaphragms.

FIG. 7 illustrates excitation light paths in a mirrored will during the readout process.

FIG. 8 shows how excitation light bounces in the waveguide built around the through-well during the readout process.

FIG. 9 provides a top view (FIG. 9a) and a side view (FIG. 9b) of the discrete cylinder embodiment of the 3-dimensional external probe carrier.

FIG. 10 illustrates one arrangement that may be used for binding samples to probes in the discrete cylinder embodiment of the 3-dimensional external probe carrier.

FIG. 11 schematically demonstrates the paths followed by excitation light in the readout process of the 3-dimensional external probe-carrier embodiment, without a central metal core (a) and with a central metal core (b).

FIG. 12 shows a method for identification and tracking of fiber identities after a fiber bundle is made for the 3-dimensional external probe-carrier embodiment.

FIG. 13 illustrates using guide plates to keep fibers in an orderly matrix in the bundle in the 3-dimensional external probe-carrier embodiment.

### DESCRIPTION OF THE INVENTION

The invention relates to a three-dimensional probe carrier. The description below is for two embodiments, a 3-dimensional internal probe-carrier embodiment and a 3-dimensional external probe-carrier embodiment. The invention described in this disclosure is relevant to all biological or chemical porous probe-bearing substrates, including DNA, protein, chemical compound porous probe-bearing substrates. Solely for the reason of convenience, four specific applications of the porous probe-bearing substrate are described as examples. These include protein microarray, PCR microarray, chemical compound microarray and immobilized DNA microarray. The first three are preferably fluid-based microarrays.

In addition to the four application areas mentioned above, the technique can also be used to produce porous probe-bearing substrates of a wide ranging biological and chemical materials which include but not limited to deoxyribonucleic acids (DNA), ribonucleic acids (RNA), synthetic polynucleotides, antibodies, proteins, polypeptides, peptides, lectins, modified polysaccharides, synthetic composite macromolecules, functionalized nanostructures, synthetic polymers, modified/blocked nucleotides/nucleosides, modified/blocked amino acids, fluorophores, chromophores, ligands, chelates, haptens and drug compounds. The samples being deposited on the porous probe-bearing substrate using the technology disclosed here can take or be carried by any physical form that can be transported through a capillary. These include but are not limited to fluid, gel, paste, bead, powder and particles suspended in liquid.

A “probe,” as used herein, is a set of copies of one type of molecule or one type of molecular structure. The set may contain any number of copies of the molecule or multimolecular structure. “Probes,” as used herein, refers to more than one such set of molecules or multimolecular structures. The molecules or multimolecular structures may be chemical compounds, polynucleotides, oligonucleotides, polypeptides, oligosaccharides, polysaccharides, antibodies, cell receptors, ligands, lipids, cells, or combinations of these structures, or any other structures to which samples of interest or portions of samples of interest will bind or interact with specificity. In PCR microarrays, in particular, a probe can be a unique mixture of primers (short, synthetic pieces of single-stranded DNA that exactly match and flank, from each side of the sequence, the stretch of DNA to be amplified), deoxynucleotide triphosphates (dNTPs, the building blocks of DNA), buffer and a heat-resistant enzyme (polymerase). For quantitative PCR microarrays, a probe includes the mixture above plus a fluorigenic signal oligonucleotide.

As used herein, “polynucleotide” means a polymeric form of nucleotides of any length, which contain deoxyribonucleotides, ribonucleotides, and/or their analogs. The terms a “polynucleotide” and “nucleotide” as used herein are used interchangeably. Polynucleotides may have any three-dimensional structure, and may perform any function, known or unknown. The term “polynucleotide” includes double-, single-stranded, and triple-helical molecules. Unless otherwise specified or required, any embodiment of the invention described herein that includes a polynucleotide encompasses both the double-stranded form and each of two complementary single-stranded forms known or predicted to make up the double stranded form.

The following are non-limiting examples of polynucleotides: a gene or gene fragment, exons, introns, mRNA, tRNA, rRNA, ribozymes, cDNA, recombinant polynucleotides, branched polynucleotides, plasmids, vectors, isolated DNA of any sequence, isolated RNA of any sequence, nucleic acid probes, and primers. A polynucleotide may comprise modified nucleotides, such as methylated nucleotides and nucleotide analogs. Analogs of purines and pyrimidines are known in the art, and include, but are not limited to, aziridinylcytosine, 4-acetylcytosine, 5-fluorouracil, 5-bromouracil, 5-carboxymethylaminomethyl-2-thiouracil, 5-carboxymethyl-aminomethyluracil, inosine, N6-isopentenyladenine, 1-methyladenine, 1-methylpseudouracil, 1-methylguanine, 1-methylinosine, 2,2-dimethylguanine, 2-methyladenine, 2-methylguanine, 3-methylcytosine, 5-methylcytosine, pseudoruacil, 5-pentynyluracil and 2,6-diaminopurine. The use of uracil as a substitute for thymine in a deoxyribonucleic acid is also considered an analogous form of pyrimidine.

If present, modification to the nucleotide structure may be imparted before or after assembly of the polymer. The sequence of nucleotides may be interrupted by non-nucleotide components. A polynucleotide may be further modified after polymerization, such as by conjugation with a labeling component. Other types of modifications included in this definition are, for example, “caps”, substitution of one or more of the naturally occurring nucleotides with an analog, internucleotide modifications such as, for example, those with uncharged linkages (e.g., methyl phosphonates, phosphotriesters, phosphoamidates, carbamates, etc.) and with charged linkages (e.g., phosphorothioates, phosphorodithioates, etc.), those with intercalators (e.g., acridine, psoralen, etc.), those containing chelators (e.g., metals, radioactive metals, boron, oxidative metals, etc.), those containing alkylators, those with modified linkages (e.g., alpha anomeric nucleic acids, etc.), as well as unmodified forms of the polynucleotide(s).

Further, any of the hydroxyl groups ordinarily present in the sugars may be replaced by phosphonate groups, phosphate groups, protected by standard protecting groups, or activated to prepare additional linkages to additional nucleotides or to solid supports. The 5′ and 3′ terminal OH groups can be phosphorylated or substituted with amines or organic capping groups moieties of from 1 to 20 carbon atoms. Other hydroxyls may also be derivatized to standard protecting groups.

Polynucleotides can also contain analogous forms of ribose or deoxyribose sugars that are generally known in the art, including, but not limited to, 2′ -O-methyl-, 2′ -O-allyl, 2′ -fluoro- or 2′ -azido-ribose, carbocyclic sugar analogs, -anomeric sugars, epimeric sugars such as arabinose, xyloses or lyxoses, pyranose sugars, furanose sugars, sedoheptuloses, acyclic analogs and abasic nucleoside analogs such as methyl riboside. As noted above, one or more phosphodiester linkages may be replaced by alternative linking groups. These alternative linking groups include, but are not limited to, embodiments wherein phosphate is replaced by P(O)S (“thioate”), P(S)S (“dithioate”), ″(O)NR2 (“amidate”), P(O)R, P(O)OR′ , C(O)CH2 (“formacetal”), in which each R or R′ is independently H or substituted or unsubstituted alkyl (1-20 C) optionally containing and ether (—O—) linkage, aryl, alkenyl, cycloalky, cycloalkenyl or araldyl. Not all linkages in a polynucleotide need be identical.

Substitution of analogous forms of sugars, purines and pyrimidines can be advantageous in designing a final product, as can alternative backbone structures like a polyamide backbone.

The terms “polypeptide”, “oligopeptide”, “peptide” and “protein” are used interchangeably herein to refer to polymers of amino acids of any length. The polymer may be linear or branched, it may comprise modified amino acids, and it may be interrupted by non-amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids, etc.), as well as other modifications known in the art. Polypeptides can occur as single chains or associated chains.

3-Dimensional Internal Probe Carrier

A. The apparatus

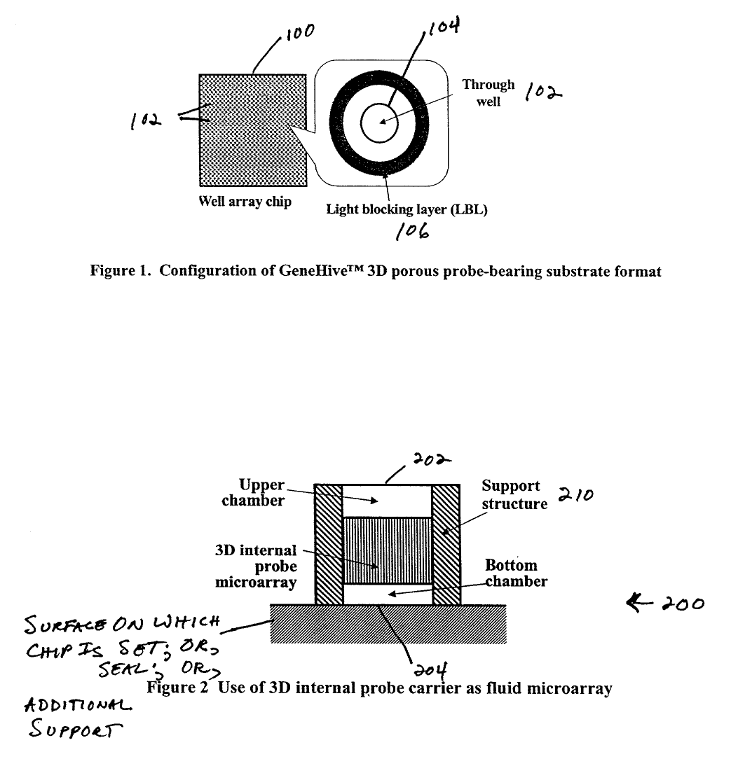

The basic configuration of the 3-dimensional internal probe-carrier embodiment, as shown in FIG. 1, involves a substrate 100 with a large number of discrete through wells or holes 102. Each well is individually identifiable by its position on the substrate 100 and has specific probe molecules either contained within the space of the well or attached to the inner sidewall 104 of the through well. A light blocking layer 106 is preferably constructed around each well to prevent the fluorescent signal emitted from one well interferes that from adjacent ones. The material for 3-dimensional internal probe-carrier substrate can be glass, silica, polymer, plastic, ceramics or even metal.

The thickness of the 3D internal probe carrier chip 200 can range from 100μm to 20mm or more and can stand alone or attached to a supporting structure. In general, the chip is thicker when it is used to perform fluid phase tests. FIG. 2 shows a 3D fluid probe carrier chip 200 with a support structure 210. A relatively thick substrate allows the wells to be long and narrow. In addition, inner surfaces of the wells may be made hydrophilic while the top 202 and bottom 204 surfaces of the substrate are treated to become hydrophobic. For example, the top 202 and bottom 204 surfaces may be silanated, and thus rendered hydrophobic, while protecting the inner surface of the wells from silanation. Fluids are drawn into the wells and held within by the capillary force. Small openings at each side ensures that fluid evaporation is kept to minimum. Additional protective films such as adhesive polymer or metal films can be attached to the top 202 and bottom 204 surface of the substrate to further reduce evaporation during longer-term handling or storage.

The volume and sidewall surface area of each well in the chip can be expressed as π²h and 2πrh, respectively, where r is the radius of the well and h is the thickness of the chip and thus the height of the inner surface of a through well. In a typical example where the porous probe-bearing substrate has 100μm probe pitch with r=30 μm and h=3mm, the volume in each well for fluid phase reaction is about 8 nl. The surface area available for probe immobilization is 0.6 mm² which is 200 times more than that in the conventional planar 2-dimensional microarray format.

B. Fabrication

This invention includes at least two methods to fabricate the 3-dimensional internal probe-carrier with high throughput at a low-cost, namely, “unitary bundle” and “assembled bundle” methods.

1. Unitary Bundle Method

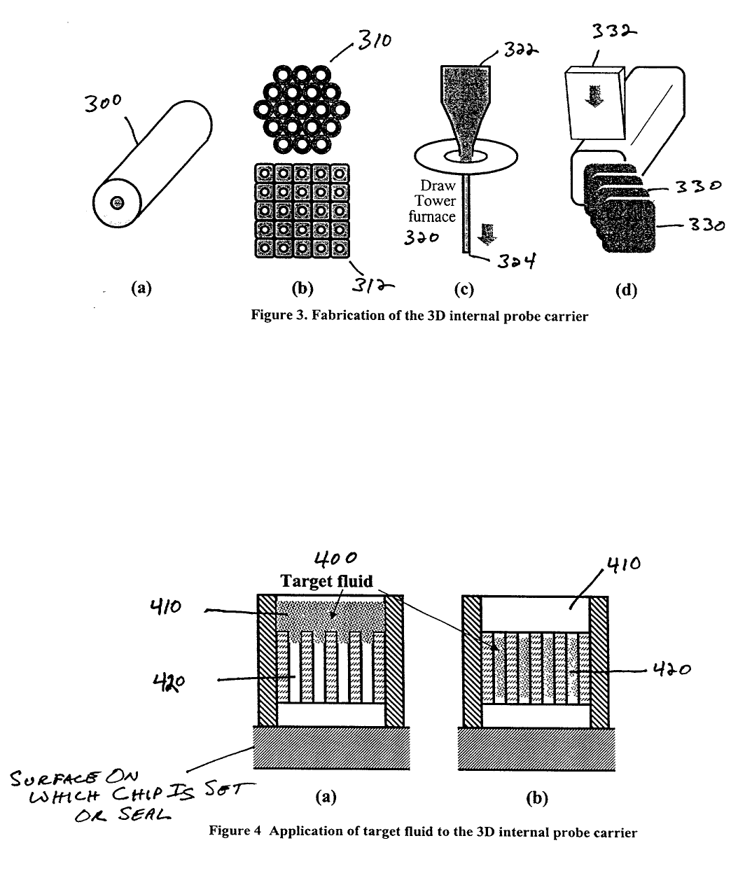

In this method, the 3-dimensional internal probe-carrier is fabricated by the following steps illustrated in FIG. 3.

Step 1: Tube preforms 300 are made by methods well-known in the optical fiber field.

Step 2: Many preforms as described in Step 1 are stacked up to form an orderly matrix 310. The matrix can take a honeycomb 310 or a chessboard 312 pattern depending on preform shape and stacking method (FIG. 3b). The stack may be heated in a furnace to near the preform melting point to weld or fuse the preforms together.

Step 3: The preform stack is extruded on an extruder or draw tower. After extrusion, the outer diameter of the stack is reduced and the diameter of the individual preforms is reduced in proportion. The tubing matrix in the stack is preferably shrunken to a pitch equal to that of a standard microtiter plate (96, 364 or 1456 wells).

Step 4: Many extruded preform bundles are stacked and welded together as described above to form a large stack comprising a large number of reduce-sized preforms.

Step 5: Finally, the large stack is further extruded on an extruder or draw tower 320, as shown in FIG. 3c. The result is a unitary capillary bundle. At one end 322, a small portion of the bundle is left large and substantially unshrunken. The cross-section of the tubing matrix at its large ends 322 preferably has the same pattern and spacing as wells in a standard microtiter plate. At the other end 324, for a vast majority of the length (up to kilometers) of the bundle, the tubing matrix is proportionally reduced to a size and pitch equal to the desired size and pitch of the 3-dimensional internal probe-carrier.

Step 6: Where necessary, the inner surface of each capillary is treated to become hydrophilic in the case of fluid phase microarray. Alternatively, the inner surfaces can be treated for probe immobilization.

Step 7: To associate probes with the substrate, probe fluids are transferred from a standard microtiter plate into the larger end of the unitary capillary bundle and are driven by pressure or pulled by capillary force to fill the entire length of the bundle.

Step 8: For fluid microarray applications, probe fluids are preferably frozen inside the capillaries. For immobilized probe arrays, the probes molecules will attach to the treated inner surfaces of the capillaries in the bundle.

Step 9: The unitary bundle is cut into chips 330 (FIG. 3d). If the bundle is made of polymer or plastic, the cutting can be performed using a tomography cutting tool 332 with diamond-edged blades. If the bundle is made of silica or glass, it is preferably cleaved or cut with a diamond saw and then polished.

Step 10: The array will remain frozen in shipment and storage and only be thawed before usage. More preferably, for 3D probe carrier chip with DNA, chemical compound or antibody probes, the cut chips can be thawed and the residual water dried out at the factory before delivery to users (e.g., lyophilized). The probe molecules will attach but not bind to the inner wall of the well, thus can be dissolved back into water when the target fluid is applied to the carrier chip at user site. For protein based probes which denature when dried, the chips can be placed in a vacuum chamber under freezing conditions and lyophilized. The procedure draws out water directly without a thawing process and has proved capable of preserving the efficacy of the protein.

2. Assembled Bundle Method

This fabrication method comprises the following steps:

Step 1: The same as Step 1, above.

Step 2: The preform is drawn directly into a very long (up to tens of kilometers), uniform and flexible capillaries with outer diameter similar to the desired pitch in the 3-dimensional internal probe-carrier.

Step 3: The same as Step 6, above.

Step 4: The very long capillary is cut into many capillaries of equal length.

Step 5: Each probe is filled into an individual capillary.

Step 6: The same as Step 8, above.

Step 7: Frozen capillaries are bundle together by epoxy or other means.

Step 8: The same as Step 9, above.

Step 9: The same as Step 10, above.

C. Probe/Target Reactions

FIG. 4 shows the use of the 3D internal probe carrier in fluid phase microarray applications. The target fluid 400 is applied to the top surface of the carrier chip (4a). The volume of target fluid is controlled to be less than the total vacant volume in all wells 410 combined. The target fluid 400 can then be completely drawn into individual capillaries 420 by capillary force (4b). Probes originally attached to the inner surfaces of the wells are dissolved into the fluids, allowing biological or chemical reactions to occur in fluid phase. As illustrated in FIG. 2, the support structure 210 of a fluid probe bearing carrier has to suspend the carrier chip 200 so that its top 202 and bottom 204 surface do not contact other objects. This prevents the creation of unwanted capillary force that may pull fluid out from wells. Because the capillary forces in the wells always dominate, fluid will not flow outward from the well structure. Hence probe in one well cannot flow to the adjacent wells, eliminating the possibility of cross-contamination in the fluid array.

For PCR microarray applications, the entire chip can be placed into a temperature controlled chamber to conduct the required thermal cycling. Because of the small chip size, the thermal mass of the chamber will be very small, which leads to faster and more precise PCR process.

If the probes are in fluid form before the application of the target fluid 400, there may be air bubbles trapped between the target and probe fluids, which prevents the biological or chemical reactions from occurring. In this situation, ultrasound administered at a suitable power and frequency may be used to break up the bubble.

When the 3D internal probe carrier described above is used to carry immobilized probes. A probe-target binding process typically occurs during the probe-target reactions, which is normally termed “hybridization”.

In the usual probe-target binding process, typically polynucleotide hybridization in current microarrays, a molecular strand in a target fluid moves in random motion due to thermal energy. Binding (e.g., hybridization) occurs when it meets a complementary probe strand, which is optimally attached to the sidewall in one of the through wells. Therefore, in order to increase the efficiency of the binding process, target molecules should be “driven” to “visit” as many wells as possible within a fixed length of time. This invention provides a number of methods and apparatus, which can be used alone or in combination to improve the hybridization efficiency:

The substrate can be completely submerged in the target fluid, so that the capillary force will not hinder the movement of molecules. In this embodiment, probes are preferably attached to the walls forming the hole.

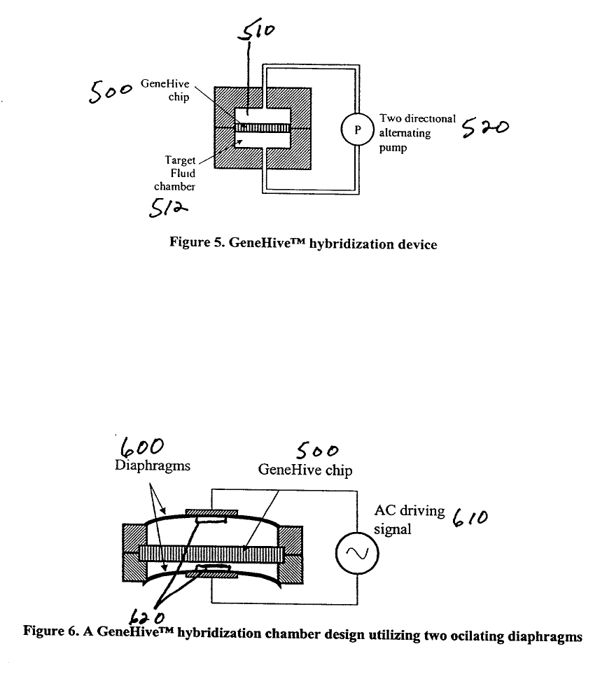

A binding enhancement device, as shown in FIG. 5, can be used to actively pump 520 the target fluid 510 back and forth through the wells in the substrate of the 3-dimensional internal probe-carrier 500 which is placed within a chamber 512 containing the target fluid 510.

FIG. 6 shows a particular design where a pair of diaphragms 600 driven by 180° out of phase signal provides the pumping action. Miniature propellers 620 in the upper and lower fluid chambers driven by an AC driving signal 610, further help to move the fluid through the multiple wells of the 3-dimensional internal probe-carrier 500. In one embodiment, smaller well diameters force the target molecules to flow closer to the sidewall, which increase binding efficiency.

D. Readout

When the space inside a well contains a probe, fluorescent material such as fluorescent tags on target molecules can be directly excited by illuminating the excitation light in the well.

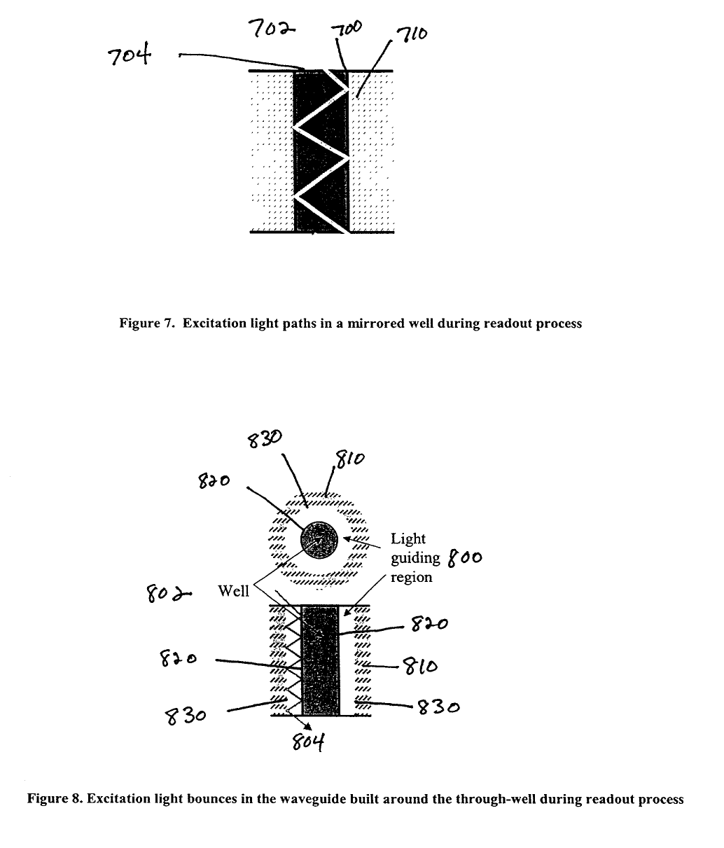

When multiple wells are excited at the same time, a light blocking layer (LBL), as shown in FIG. 1 has to be constructed around each well to prevent the fluorescent signal emitted from one well interferes that from another. This LBL can be constructed by coating a layer of emission absorption material (EAM) on the tube preforms (shown in FIG. 3a) before they are stacked and drawn into porous probe carriers. In a preferred embodiment, a layer of highly reflective coating, either metal or dielectric, is coated on the inner wall of each well. It not only serves as a LBL but also enhances the efficiency of fluorescent excitation. In normal microarray, the excitation light pass through the probe only once and only a small fraction of the emitted fluorescent light is collected by the reader. In a well with mirrored wall, as shown in FIG. 7, the excitation light zig-zag through the probe many times before exiting the well. In addition, the emitted fluorescent light is guided towards the openings on each end of the well facilitating a much more efficient signal.

When the probe is attached to the inner wall of the through well, , direct excitation is very inefficient due to the vertical angle of the side wall. This invention provides methods and apparatus to allow the 3-dimensional internal probe-carrier to be read with a standard probe-bearing substrate scanner.

As illustrated in FIG. 7, a reflective layer 700 can be coated on the sidewalls 710 of the wells, which creates an optical waveguide in the through-well. Excitation light 702 is coupled into the well waveguide at a large numerical aperture. It bounces along the side wall 710 of the well as illustrated to excite the fluorescent label. The fluorescent light is then trapped by the sidewalls 710 of mirror-walled well and guided towards opening 704 of the well, which can be observed by a standard microarray reader. The reflective coating on the side wall can be made of multiple dielectric thin films or metal deposited on the side wall.

Another embodiment of the invention is illustrated in FIG. 8. A light guiding region 800 around the through well 802 is made to have higher optical refractive index than outer region 810. Because the refractive index inside the well is near 1.0 (air), also less than the refractive index of the ring-shaped light-guiding region 800 around the well, an optical waveguide is created around the well 802. Excitation light 804 coupled into the ring-shaped waveguide will bounce between the well sidewall 820 and the other boundary 830 of the waveguide. When the light is reflected by the sidewall, the evanescent field of the light excites the fluorescent label of the prove-sample complex attached to the inner surface. Part of the fluorescent light is trapped by the waveguide and guided to the top or bottom surface of the substrate, where it can be observed by a standard microarray reader.

This light guiding layer can be manufactured at tube preform stage using the modified chemical vapor deposition (MCVD) process well known in optical fiber community.

II. 3-Dimensional External Probe Carrier

A. Apparatus

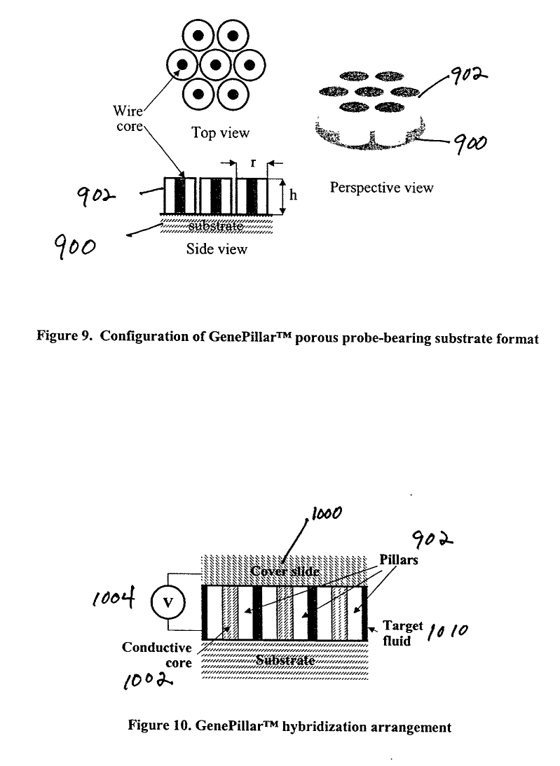

The basic configuration of the 3-dimensional external probe-carrier embodiment, as shown in FIG. 9a, involves a substrate 900 with a large number of discrete pillars or stumps 902. Each pillar 902 is individually identifiable by its position on the substrate 900 and has specific probe molecules attached to the side wall of the pillar 902. The material of the 3-dimensional external probe-carrier substrate can be glass, silica, polymer, plastic, ceramics or even metal.

Similar to the 3-dimensional internal probe-carrier embodiment, the surface area of the side wall can be expressed as 2πrh, where r is the radius of the well and h is the height of the pillar. In comparison, the binding surface area is π² in a conventional 2D microarray with equivalent probe density. The ratio of the surface area on which probes can be attached to a 3D porous probe-bearing substrate to the surface area on which probes can be attached for a 2D microarray is 2h/r. In a typical example where the porous probe-bearing substrate has 100μm probe pitch with r =40μm and h=120μm, the area available for probe binding in the proposed cylindrical 3-dimensional external probe-carrier embodiment is 6 times as much as that in the conventional 2D microarray format.

The cylinder embodiment of the 3-dimensional external probe-carrier can also take the shape of a shot pin or rod 910, as shown in FIG. 9b. This allows them to be packaged into a matrix on a flat substrate 912 with the same pattern and pitch as a standard microtiter plate to be dipped into the wells of the microtiter plate to perform multi-sample binding in parallel.

B. Use

1. Sample binding

The 3-dimensional external probe-carrier can be used to bind sample, for example to hybridize a polynucleotide sample to polynucleotide probes, almost the same way as a conventional 2D microarray. As shown in FIG. 10, a cover slide 1000 can be directly applied on the top of the array because the probes are attached to the side walls, not the tips of the 3-dimensional external probe-carriers (pillars) 902. Traditional thermal, acoustic and vibration methods can be used to encourage target molecules to move from pillar to pillar to enhance binding (e.g., hybridization) efficiency. In addition, if the samples to be analyzed are charged, a conductive core 1002 can be embedded in each pillar. This can be used, for example, with DNA, which carries a negative charge. The core can be a conductive metal or alloy in the form of one or more wires or rods placed within the pillar along the pillars longitudinal axis. By applying a positive polarity voltage to the core inside the pillar, negatively charged DNAs are attracted to the pillar surface, which increases local concentration, thus improves binding opportunities. On the other hand, a negative polarity voltage can be applied to the core, which pushes nonspecifically bonded molecules away from the binding sites thus increasing the binding specificity of the hybridization. By applying AC voltage 1004 to the pillar, the hybridization efficiency can be improved.

In addition, because the pillars can be tightly packed, the gap between pillars is very small. This reduces the volume of target fluid 1010 required for binding.

2. Readout

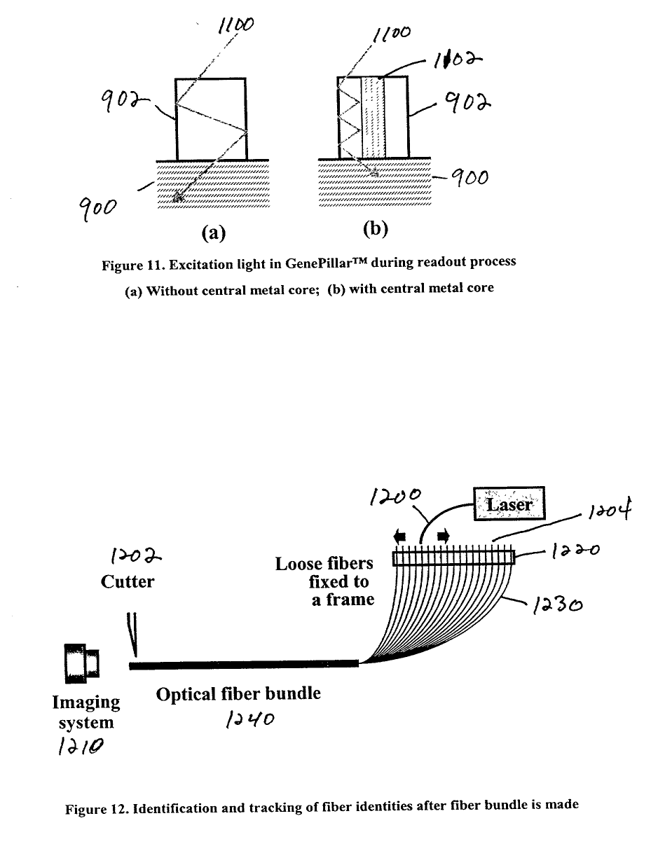

When the pillar is made of silica/glass or transparent polymer/plastic, it becomes a natural optical waveguide because its refractive index is larger than the surrounding medium, air. As shown in FIG. 11, the excitation light 1100 coupled into the 3-dimensional external probe-carrier 902 is bounced many times on the side wall before exiting to the supporting substrate 900. An optional central metal core 1002 further increases the number of times the excitation light 1100 bounces while traveling though 3-dimensional external probe-carrier 902. The evanescent field of the reflected light excites the fluorescent label of the sample-probe complexes attached to the side wall surface. Part of the excited fluorescent light will be trapped inside the 3-dimensional external probe-carrier and guided towards the upper or lower surface, where it can be observed by a conventional microarray.

C. Fabrication

The 3-dimensional external probe-carrier can be fabricated with high throughput at a low-cost with the following steps as illustrated in FIG. 12:

Step 1: Fabricate “air-cladding” optical fibers. Normal optical fiber used in telecommunication has a cladding layer of lower refractive index surrounding the core, where light is guided. In this application, air is used as the cladding material. The fiber 1230 can be drawn from pure glass, silica or polymer rods. Such fiber are available on the market for sensing applications. In order to implement the binding enhancement by alternating polarity described above, the fiber 1230 can be drawn out of conductive polymer or alternatively, the fiber 1230 can also be fabricated by coating a layer of light guiding polymer on to a thin metal thread.

Step 2: The fabricated fibers are cut into many equal length sections and surface treated for probe attachment. If the probes are polynucleotides, methods and materials for derivatization of solid phase supports for the purpose of immobilizing polynucleotides are known to those skill in the art and described in, for example, U.S. Pat. No. 5,919,523, which is incorporated herein by reference. The polynucleotide probes of the invention are affixed, immobilized, provided, and/or applied to the surface of the solid support using any available means to fix, immobilize, provide and/or apply polynucleotides at a particular location on the solid support. The various species may be placed at specific sites using ink jet printing (U.S. Pat. No. 4,877,745, which is incorporated herein by reference), photolithography (See, U.S. Pat. Nos. 5,919,523, 5,837,832, 5,831,070, 5,770,722 and 5,593,839, all of which are incorporated herein by reference), silk printing, offset printing, stamping, mechanical application with micropipets using an x-y stage or other rastering technique, or any other method which provides for the desired degree of accuracy and spatial separation in placing the bound component. The polynucleotide primers may also be applied to a solid support as described in Brown and Shalon, U.S. Pat. No. 5,807,522 (1998), which is incorporated herein by reference. Additionally, the primers may be applied to a solid support using a robotic system, such as one manufactured by Genetic MicroSystems (Woburn, Mass.), GeneMachines (San Carlos, Calif.) or Cartesian Technologies (Irvine, Calif.).

Step 3: Individual fibers are soaked in specific probe fluids, respectively, to allow attachment of probe molecules to the outer surface of the fiber.

Step 4: Fibers with probes attached are dried and coated with a thin buffer layer such as wax, which could be removed later without damaging the probe molecules.

Step 5: Many fibers with this buffer layer are molded together using a buffer material, such as wax, to form a bundle 1240 in one end and left loose at the other end or optionally attached to a frame 1220.

Step 6: The bundle 1240 is cut into thin slides using a tomography tool 1202. Because a large number of fibers are bundled together at random, the identity of each fiber in the bundle may be lost. Initially, the fiber IDs in the bundle 1240 can be re-established by launching light 1200 one-by-one, into fibers at the loose end 1204, where their individual ID is still known and observe the exit point of the light at the bundled end. An example of an apparatus to carry out this fabrication is shown in FIG. 12. Because the cut slide is very thin, once the fiber ID is established, its position changes only slightly from slide to slide. An imaging system 1210 can be installed to on-line monitor the small change in fiber position from slide to slide, thus tracking the identity of the pillar cut from the bundle.

An alternative method of fabrication is the following:

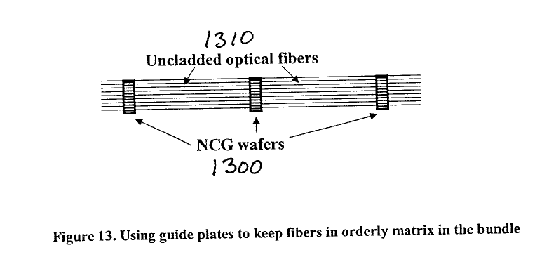

Step 1: Guide plates 1300 made of Nano-Channel Glass wafers (NCG) (Tonucci, R. J., Justus, B. L., et. al., 1992, Science 258; 783-785, which is incorporated herein by reference) can be used in regular intervals to hold the fibers (without wax coating) 1310 in the bundle into an orderly matrix, as shown in FIG. 13.

Step 2: A suitable buffer material such as wax is then poured around and in the bundle to hold the relative positions fixed among fibers.

Step 3: The bundle is cut into thin chips. Because the fibers are in an orderly matrix and can readily be identified by its position in the matrix, no re-identification is required.

Step 4: The slide is bound to a supporting substrate by epoxy or weld.

Step 5: The buffer material is removed to expose the side walls of the pillars.

### Conclusion

The inventions disclosed herein provide a novel 3-dimensional arrangement of probes on a probe carrier with concomitant increases in the economy of reagents, compactness and readability of the probe carriers. Novel methods of fabricating probe carriers are also disclosed. Techniques for hybridization assays and reading the results of such assays are also presented.

All publications and patent applications mentioned in this specification are incorporated herein by reference to the same extent as if each individual publication or patent application were specifically and individually indicated to be incorporated by reference.

The above description is illustrative and not restrictive. Many variations will be apparent to those skilled in the art upon review of this disclosure. The scope of the invention should not be determined with reference to the above description, but instead should be determined with reference to the appended claims and the full scope of their equivalents.

What is claimed is:

1. A three dimensional probe array comprising a substrate formed of a substrate material and having multiple probe wells, each of said probe wells having a top surface, a bottom surface, an inner sidewall, an opening in said top surface, and an opening in said bottom surface; wherein a first probe well of said multiple probe wells contains probes, wherein the first probe well comprises a first light-conducting material and a second material, and wherein said first light-conducting material and said second material are configured such that a light beam launched into the opening of the first probe well in said top surface is transmitted by said first light-conducting material and exits the opening of said first probe well in said bottom surface.

2. A three dimensional probe array according to claim 1 wherein said first light-conducting material has a first refractive index and said second material has a second refractive index that is less than the first refractive index such that the first probe well forms a light-conducting waveguide.

3. A three dimensional probe array according to claim 2 wherein the first light-conducting material of the first probe well comprises a liquid in fluid contact with the inner sidewall of the first probe well and the second material comprises said sidewall.

4. A three dimensional probe array according to claim 2 wherein the first light-conducting material comprises a first portion of the first probe well that includes the inner sidewall and the second material comprises a portion of the substrate.

5. A three dimensional probe array according to claim 2 wherein the first light-conducting material comprises a first portion of the first probe well that includes the inner sidewall and the second material comprises a second portion of the first probe well bounded by said first portion and an outer sidewall of the first probe well.

6. A three dimensional probe array according to claim 2 wherein said first light-conducting material comprises silica doped with an impurity that increases the refractive index of silica and wherein the second portion comprises silica having a lower refractive index than said first portion.

7. A three dimensional probe array according to claim 2 wherein the first light-conducting material comprises a reflective material.

8. A three dimensional probe array according to claim 7 wherein the reflective material comprises multiple dielectric thin films deposited on the inner sidewall of the first probe well.

9. A three dimensional probe array according to claim 7 wherein the reflective material comprises metal deposited on the inner sidewall of the first probe well.

10. A three dimensional probe array according to claim 1 wherein the first probe well comprises a capillary.

11. A three dimensional probe array according to claim 1 wherein the substrate comprises multiple tubes having a length and adhered to one another along the length and along outer sidewalls of the tubes.

12. A three dimensional probe array according to claim 1 wherein each of said multiple probe wells individually comprises a capillary.

13. A three dimensional probe array according to claim 12 wherein each of said multiple probe wells forms a portion of a unitary array of said probe wells.

14. A three dimensional probe array according to claim 12 wherein each of said multiple probe wells forms a portion of a bundle of said probe wells.

15. A three dimensional probe array according to claim 1 wherein the multiple probe wells comprise multiple capillaries bound together to form a bundle.

16. A three dimensional probe array according to claim 15 wherein said bundle is an unordered bundle.

17. A three dimensional probe array according to claim 15 wherein said bundle is an ordered bundle.

18. A three dimensional probe array according to claim 16 wherein the substrate comprises a binder that holds the capillaries together.

19. A three dimensional probe array according to claim 18 wherein the binder comprises at least one member of the group consisting of epoxy, silica, a metal band, a plate having through-holes through which the capillaries pass, and a plate to which each of the bottom surfaces of the probe wells is attached.

20. A three dimensional probe array according to claim 1 wherein the opening in the top surface and the opening in the bottom surface of the first probe well form a portion of a channel through the first probe well, which channel has a length and which channel has a cross-sectional area that is essentially constant along the length of the channel.

21. A three dimensional probe array according to claim 1 wherein the opening in the top surface and the opening in the bottom surface of the first probe well form a portion of a channel through the first probe well, which channel has a length and which channel has a cross-sectional area that decreases along the length of the channel.

22. A three dimensional probe array according to claim 1 wherein the probe array further comprises a base plate to which the bottom surface of the first probe well is attached.

23. A three dimensional probe array according to claim 1 wherein said probe wells are distributed across the substrate at a density greater than 400 probe wells per square centimeter.

24. A three dimensional probe array according to claim 1 wherein the probes comprise at least one member selected from the group consisting of biological and chemical samples.

25. A three dimensional probe array according to claim 1 wherein the probes are attached to the inner sidewalls of the probe wells.

26. A three dimensional probe array according to claim 1 wherein the probes are suspended in a liquid contained in said probe wells.

27. A three dimensional probe array according to claim 1 wherein the probe wells comprise silica.

28. An array of pillars comprising multiple pillars having proximal ends, distal ends, and sidewalls, wherein said proximal ends of said pillars are affixed to a surface of a substrate, and each of said pillars has a probe attached to the sidewalls of said pillars.

29. An array of pillars according to claim 28, wherein said surface of said substrate is planar.

30. An array of pillars according to claim 28, wherein said substrate is a rod having two ends and a sidewall, wherein said surface of the substrate comprises said sidewall.

31. An array of pillars according to claim 28, wherein said distal end of each said pillar is free of biological and chemical sample.

32. An array of pillars according to claim 28, wherein the longest axis of each said pillar is less than 200 μm.

33. An array of pillars according to claim 28, wherein said pillars are distributed across said substrate at a density greater than 400 pillars per square centimeter of substrate surface.

34. An array of pillars according to claim 28, wherein said pillars each have an electrically conductive core.

35. An array of pillars according to claim 28, wherein said pillars are light-conducting pillars.

36. A method of generating an array of capillaries comprising:
a) forming an ordered bundle of a plurality of light conducting capillaries having distal and proximal ends and a channel extending from said distal end to said proximal end, wherein each said capillary is capable of conducting light parallel to the channel, and wherein the position of the distal end and proximal end of each said capillary are known;
b) securing the proximal ends of the capillaries to form a solid mass containing the proximal ends of said capillaries, said solid mass having a facet and said proximal ends being substantially coplanar at said facet; and
c) cutting said bundle into slices, wherein said capillaries are filled with biological or chemical samples prior to cutting into said slices.

37. A method of generating an array of capillaries comprising:
a) forming a random bundle of a plurality of capillaries having distal and proximal ends;
b) securing the proximal ends of the capillaries to form a solid mass containing the proximal ends of said capillaries, said solid mass having a facet and said proximal ends being substantially coplanar at said facet; and
c) cutting said bundle into slices, wherein said capillaries are filled with biological or chemical samples prior to cutting into slices.

38. A method of generating an array of capillaries according to claim 37, wherein said capillaries are light conducting capillaries.

39. A method of generating an array of capillaries according to claim 38, further comprising registering the proximal ends of the capillaries to the distal ends of the capillaries.

40. A method of generating an array of capillaries according to claim 39, wherein said registering comprises launching light into a distal end of a first capillary, observing the light exiting a proximal end of the first capillary and recording information that correlates the distal end of the first capillary to the proximal end of the first capillary.

41. A method of generating an array of capillaries according to claim 40, wherein said biological or chemical samples bind to the sidewall of said channels of said capillaries.

42. A method of generating an array of capillaries according to claim 41, wherein said channel of each said capillary is coated with a material such that the coating reflects light within the channel.

43. A method of generating an array of capillaries according to claim 36, wherein said capillaries further comprise a first portion having a first refractive index and a second potion having a second refractive index, said second refractive index being greater than said first refractive index, wherein said first and second portion are configure such that a beam of light launched into the proximal end is transmitted along the capillary and exits the capillary at the distal end.

44. A method of generating an array of capillaries according to claim 37, wherein said capillaries further comprise a first portion having a first refractive index and a second potion having a second refractive index, said second refractive index being greater than said first refractive index, wherein said first and second portion are configure such that a beam of light launched into the proximal end is transmitted along the capillary and exits the capillary at the distal end.

45. A method of binding a target to probes in a probe array comprising:
a) providing a probe array according to claim 1; and
b) pumping a fluid containing target molecules from a first fluid reservoir through at least one probe well to a second fluid reservoir.

46. A method of binding a target to probes in capillary arrays according to claim 45, further comprising pumping said fluid from the second fluid reservoir through said probe well through which said fluid was previously pumped.

47. A method of detecting target molecules bound to probe molecules in a probe array comprising:
a) providing a probe array according to claim 1;
b) transmitting a light into a first portion of a first capillary; and
c) detecting whether target molecules are bound to the biological or chemical sample within said first capillary based upon an observable effect caused by transmitting said light into said capillary.

48. A method of manufacturing an array of pillars comprising:
a) attaching biological or chemical samples to a sidewall of a plurality of pillars having proximal and distal ends;
b) coating said pillars with a removable protective layer;
c) forming a bundle of said pillars;
d) securing the proximal ends of said pillars to form a solid mass containing the proximal ends of the pillars, said solid mass having a facet and said proximal ends being substantially coplanar at said facet; and
e) cutting said bundle into slices.

49. A method according to claim 48 wherein said bundle is a random bundle of pillars.

50. A method according to claim 48 wherein said bundle of pillars is formed by threading a plurality of said pillars through at least one guide plate.

51. A method of manufacturing an array of pillars according to claim 48, further comprising affixing each said slice to a substrate such that the proximal ends of said pillars are affixed to said substrate.

52. A method of manufacturing an array of pillars according to claim 48, wherein each said pillar comprises a material capable of transmitting a signal from the distal end to the proximal end.

53. A method of manufacturing an array of pillars according to claim 52, further comprising registering said proximal ends of the pillars to the distal ends of the pillars.

54. A method of manufacturing an array of pillars according to claim 53, wherein the act of registering the proximal ends to the distal ends comprises launching a signal into a distal end of a first pillar, observing the signal at a proximal end of the first pillar, and recording information that correlates the distal end of the first pillar to the proximal end of the first pillar.

55. A method of manufacturing an array of pillars according to claim 54, wherein said signal is light.

56. A method of manufacturing an array of pillars according to claim 54, wherein said signal is electrical.

57. A method of binding a probe to target molecules adhered to an array of pillars comprising:
a) providing an array of pillars according to claim 28; and
b) contacting the sidewalls of said pillars with a target-containing fluid.

58. A method according to claim 57, wherein said pillars further comprise an electrically conductive core and wherein efficiency of binding is enhanced by applying to said pillar core a voltage of opposite polarity to the overall charge of the target molecules.

59. A method according to claim 57, wherein said pillars further comprise an electrically conductive core and wherein efficiency of binding is enhanced by applying an alternating voltage to said pillar cores.

60. A method of binding a probe to target molecules adhered to an array of pillars according to claim 57, wherein said pillars further comprise an electrically conductive core and further comprising enhancing binding specificity by applying to said pillar cores a voltage of the same polarity as the overall charge of the non-target molecules.

61. A method of detecting binding of a probe to target molecules adhered to an array of pillars comprising:
a) providing an array of pillars comprising a plurality of pillars having proximal and distal ends, wherein said proximal ends are affixed to a substrate, and each of said pillars has a biological or chemical sample attached to the sidewalls of said pillar;
b) contacting the sidewalls of said pillars to a target molecule-containing fluid; and
c) detecting said target molecules bound to said chemical or biological samples.

62. A method of detecting binding of a probe to target molecules adhered to an array of pillars according to claim 61, wherein said pillars are light conducting pillars and wherein said detecting is performed by transmitting a light signal into a first pillar.

63. A method of detecting binding of a probe to target molecules adhered to an array of pillars according to claim 62, wherein said light signal induces target molecules bound to said first pillar to emit a second light signal.
